(19) United States
(12) Patent Application Publication
(10) Pub. No.: US 2005/0249037 A1
(43) Pub. Date: Nov. 10, 2005

(54) Wireless instrument for the remote monitoring of biological parameters and methods thereof

(76) Inventors: Daniel W. Kohn, Cambridge, MA (US); David L. Kuhn, Cave Creek, AZ (US); David William Frost, Scottsdale, AZ (US); Christopher Trask, Tempe, AZ (US)

Correspondence Address: DAVID KUHN AND DANIEL KOHN, P.O. BOX 7085, CAVE CREEK, AZ, 85327, US

(21) Appl. No.: 11/118,287

(22) Filed: Apr. 28, 2005

Related U.S. Application Data
(60) Provisional application No. 60/565,883, filed on Apr. 28, 2004.

(51) Int. Cl. G01S 1/72; A61B 5/00

(52) U.S. Cl. 367/117; 600/301; 128/903

(57) ABSTRACT

A wireless instrument and methods for the remote monitoring of a plurality of biological parameters is described. The instrument includes a means of receiving data from a plurality of sensors, a means for converting the sensor data to a digital format, a means for collating the digitised sensor data into a single digital data stream, a transmitter for modulating a carrier signal with the digital data stream to create a modulated signal suitable for wireless transmission, an antenna for generating a radiating electromagnetic field to be received by a remote receiver, and an electrical power source. In a further embodiment, an additional means is provided for including an identification code in the transmitted digital data stream. In a further embodiment, an additional means is provided for monitoring operational parameters of the instrument and the inclusion of the monitored operational parameters in the transmitted digital data stream. In a further embodiment, an additional means is provided that allows for receiving signals from a remote transmitter for the remote manipulation of the instrument. In a further embodiment, the electrical power source is rechargeable and a recharging means is provided. A method is described for encapsulating the instrument in a one-piece housing that may include additional materials for increasing the mass of the instrument.

### BACKGROUND OF THE INVENTION

It is known that the remote sensing of biological parameters is an established practice in both human and veterinary medicine. The remote sensing of single biological parameters is common practice, however the determination of the health of a subject more often requires the monitoring of a plurality of biological parameters which includes but is not limited to temperature, acidity (more commonly referred to as pH), and heart rate. The state of the art in remote sensing has advanced significantly in recent years, producing smaller and more reliable sensors, especially with respect to pH. In addition, rapid advances in the integration and miniaturisation of electronic devices has made it possible to incorporate an increasing number of functions in a small volume while at the same time requiring smaller amounts of electrical power. Furthermore, the cost of these electronic devices has decreased dramatically, making the remote monitoring of a plurality of biological parameters not only technically possible but also monetarily feasible. To date, the technical and financial constraints encountered in the monitoring of biological parameters have made it impractical to monitor more than a single parameter.

As mentioned earlier, the miniaturisation and reliability of certain biological sensors has been detrimental to the development of remote sensors for monitoring a plurality of biological parameters. A case in point is that of pH, which is an essential parameter in monitoring the health of ruminant animals, in particular dairy cows, but which requires long-term monitoring. Until recently, the measurement of pH relied upon sensors that were not only bulky but which were reliable for only short periods of time, after which they would require recalibration, which added significantly to the cost of such a system and which subsequently made such a system financially unattractive. It is now possible to obtain pH sensors that are not only considerably smaller in size but which can provide reliable measurements for periods of a year or more and at a cost that is considerably less than that of more contemporary instruments. It is well known in the dairy industry that a system that would enable the remote monitoring of both temperature and pH in dairy livestock without requiring periodic recalibration over a long period of time would offer a substantial economic savings to the industry by way of reducing the instances of loss of productivity due to a phenomenon known as acidosis, which can be so severe as to cause the death of an otherwise productive animal but which is easily prevented if the parameters of temperature and pH are monitored continuously.

This is but a single instance in the applications that are possible when two or more biological parameters are measured simultaneously, and the number of applications is enormous in breadth. The state of the art is such that a method and apparatus for performing such monitoring of a plurality of biological parameters is now practical from both a technical and monetary standpoint, therefore the present invention.

### SUMMARY OF THE INVENTION

A wireless instrument and methods for the remote monitoring of a plurality of biological parameters is described, which includes a plurality of biological parameter sensors, a means for converting the measured sensor data to a digital format, a means for collating the digitised sensor data into a single digital data stream, a transmitter for modulating a carrier signal with the digital data stream to create a modulated signal suitable for wireless transmission, an antenna, and a source of electrical power. The invention further includes a one piece moulded housing that protects the internal electronics from the monitored biological environment.

A detailed embodiment is described for converting the plurality of sensor data into digital format by way of an analogue multiplexer and an analogue-to-digital converter. A microprocessor or microcontroller is utilised for controlling the selection of the sensor data that is to be converted, collating the digitised sensor data into a digital data stream, managing the power distribution within the instrument, and a variety of additional functions. The invention further includes a transmitter and an antenna for the purpose of generating and radiating a modulated wireless signal that is intended to be received by a remote receiver. The invention further provides for including an amplifier in the transmitter for increasing the transmitted power. The invention further provides for including an identification number in the transmitted digital data stream. The invention further provides for including data pertaining to operational parameters of the instrument in the transmitted digital data stream. The invention further provides for including a receiver for receiving signals from a remote transmitter that are used to manipulate the instrument for the purpose of performing functions such as the calibration of the sensors. The invention further provides for a rechargeable electrical power source and a means for recharging the power source.

An advantage of the present invention is that it provides a flexible platform for remotely monitoring any number of biological parameters. Another advantage is that the various sensors may be calibrated on demand without the need of disassembling the instrument. Yet another advantage is that the electrical power source may be recharged without the need of disassembling the instrument for the purpose of replacing batteries or other expendables. A further advantage is that the instrument is packaged in a one-piece housing made from inert materials that prevents contamination of the instrument by the monitored environment, prevents the instrument from making unintended contact with the monitored environment, and which is of a shape that prevents physical injury.

### BRIEF DESCRIPTION OF THE DRAWINGS

The invention is described in the schematics of FIGS. 1 to 8, in which:

FIG. 1 schematically illustrates the system of a wireless instrument for the purpose of monitoring a plurality of biological parameters;

FIG. 2 schematically illustrates the method of converting the plurality of sensor data to a digital format by way of an analogue multiplexer and an analogue-to-digital converter;

FIG. 3 schematically illustrates the method of generating a frequency modulated carrier signal;

FIG. 4 schematically illustrates the method of generating an amplified frequency modulated carrier signal;

FIG. 5 schematically illustrates the method of generating an amplitude modulated carrier signal;

FIG. 6 schematically illustrates the method of generating an amplified amplitude modulated carrier signal;

FIG. 7 schematically illustrates the method receiving a signal from a remote transmitter; and

FIG. 8 schematically illustrates the method of remotely recharging a rechargeable electrical power storage device.

### DETAILED DESCRIPTION OF THE PREFERRED EMBODIMENTS

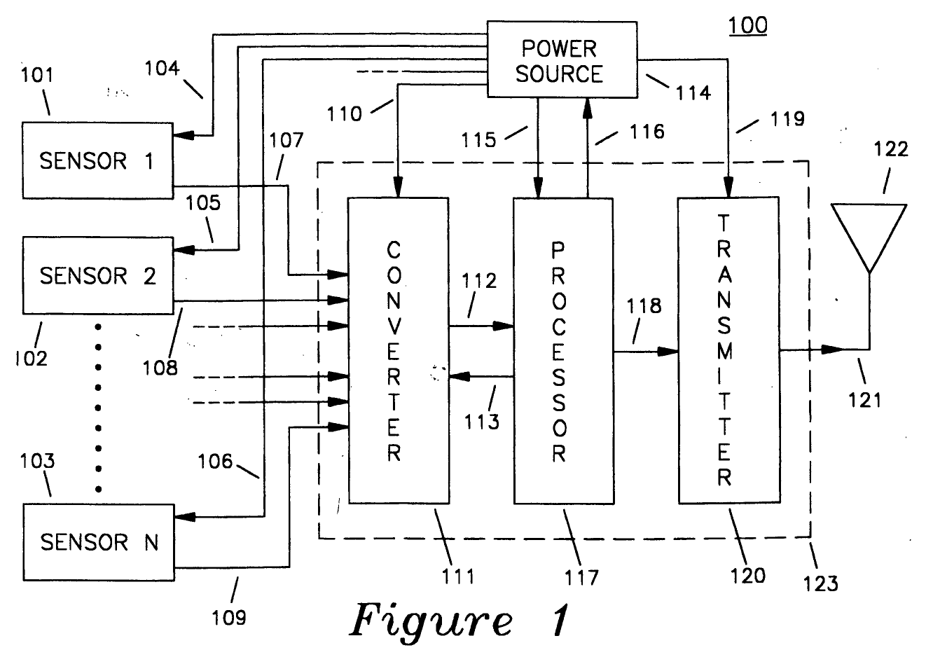

Referring to FIG. 1, the present invention of a wireless instrument for monitoring a plurality of biological parameters is described in schematic form. A series of sensors beginning with a first sensor 101, a second sensor 102, and ending with a last sensor 103 each measure their respective biological parameters and generate signals that represent the measure of their respective biological parameters. The first sensor 101 generates a first signal 107, the second sensor 102 generates a second signal 108, and the last sensor 103 generates a last signal 109. In addition to the measurement of biological parameters, one or more sensors may be used for the monitoring of one or more performance parameters of the wireless instrument itself. The first signal 107, second signal 108, and last signal 109 are then conducted to a converter 111 which selects the signals individually, converts the signals to a digital format when necessary, and generates a digital output signal 112 which is then conducted to a processor 117. Those who are familiar with the art will recognise that the converter 111 includes analogue multiplexers, analogue-to-digital converters, and digital multiplexers in combinations that are needed to select the plurality of sensor data individually, convert them to digital form when necessary, and produce a single digital output signal. Processor 117 controls the selection of sensor data to be conducted from converter 111 to processor 117 as output signal 112 by way of control signal 113.

Processor 117 collects the various digitised sensor data from converter 111, which it then processes to form an output digital data stream 118 which is then conducted to transmitter 120. The output digital data stream 118 may also contain an identification number. The output digital data stream 118 may also comprise some means of error correction. Transmitter 120 then uses the output digital data stream 118 to generate a modulated radio frequency (RF) output signal 121 which is then conducted to an antenna 122 which generates a radiating electromagnetic signal that is received by a remote receiver.

Electrical power for the wireless instrument is provided by the power source 114, which may be a fixed battery or a rechargeable storage device. Referring again to FIG. 1, from power source 114 sensor 101 receives electrical power 104, sensor 102 receives electrical power 105, sensor 103 receives electrical power 106, converter 111 receives electrical power 110, processor 117 receives electrical power 115, and transmitter 120 receives electrical power 119. The regulation and distribution of electrical power from power source 114 to the various functions may be controlled by processor 117 by way of control signal 116.

Those who are familiar with the art will recognise that the processor 117 includes a Read Only Memory (ROM), a Random Access Memory (RAM), a clock oscillator, and all other functions that have come to be associated with highly integrated programmable digital devices commonly referred to as microcontrollers. It will also be recognised by those who are familiar with the art that certain microcontrollers further include analogue multiplexers and analogue-to-digital converters, which makes it possible to provide the converter 111 and the processor 117 in a single device.

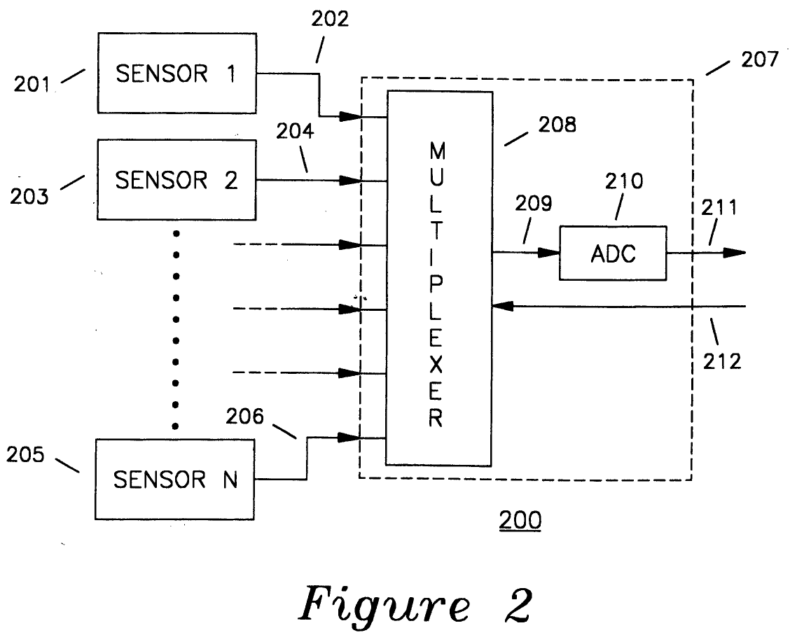

As was stated earlier, the converter 111 includes analogue multiplexers, analogue-to-digital converters, and digital multiplexers in combinations that are needed to select the plurality of sensor data individually, convert them to digital form, and produce a single digital output signal. A common form for the converter 111 is illustrated schematically in FIG. 2. Here, the converter 207 consists of an analogue multiplexer 208 and a single analogue-to-digital converter 210. A first signal 202 from the first sensor 201 is coupled to the first port of the analogue multiplexer 208. A second signal 204 from the second sensor 203 is coupled to the second port of the analogue multiplexer 208. Finally, a last signal 206 from the last sensor 205 is coupled to the last port of the analogue multiplexer 208. A control signal 212 controls the analogue multiplexer 208 to select one of the analogue signals to form signal 209 which is coupled to the input of the analogue-to-digital converter 210 which then produces the digital output signal 211. As was stated earlier, the sensors 201, 203, and 205 may be a combination of biological parameter monitoring sensors and instrument monitoring sensors.

Those who are familiar with the art readily understand that not all circumstances of converting a plurality of sensor data individually to a digital signal can be accomplished by the converter of FIG. 2 alone. Situations exist wherein one or more sensors may have a digital signal output while others have an analogue signal output. In such situations, it will be necessary to perform the overall function of converting each sensor signal individually to a single digital signal by making use of a combination of analogue multiplexers, digital multiplexers, and analogue-to-digital converters.

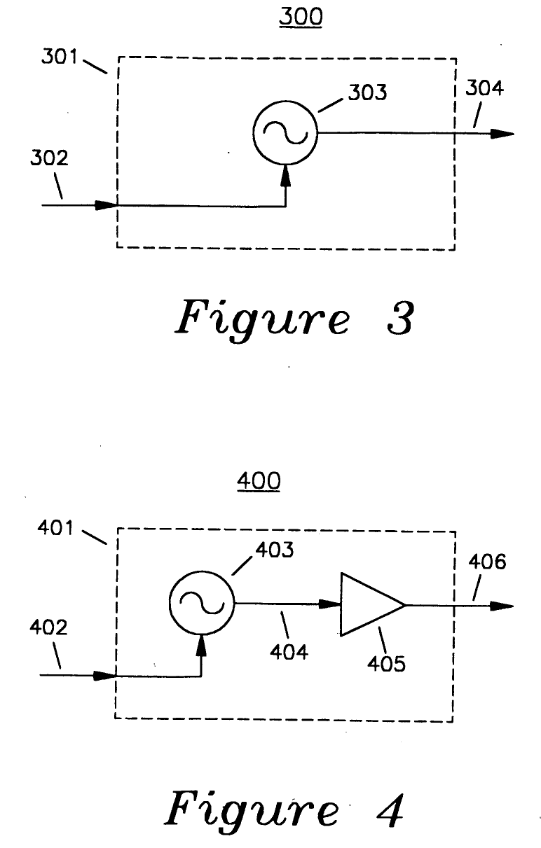

Referring back to FIG. 1, the transmitter 120 receives the digital data stream 119 from processor 117, producing a modulated RF output signal 121. In practice, such a transmitter requires at least a carrier generator for frequency modulation (FM) and binary frequency shift key (BFSK) applications. The transmitter 301 described schematically in FIG. 3 is capable of producing FM and BFSK modulated RF signals. A carrier generator 303, such as an oscillator, is shifted in frequency by a modulating signal 302, which in the present invention is the digital data stream 118 from the processor 117 of FIG. 1, to produce an FM or BFSK modulated RF output signal 304. In some applications, the RF power produced by the transmitter of FIG. 3 is insufficient to provide reliable communications, and in such instances it may be suitable to include an amplifier stage to increase the output power. Such a transmitter is shown schematically as 401 in FIG. 4 where a carrier generator 403, such as an oscillator, is modulated by a modulating signal 402, which in the present invention is the digital data stream 118 from the processor 117 of FIG. 1, producing an FM or BFSK modulated RF signal 404 which is then amplified by a power amplifier 405, producing an amplified FM or BFSK modulated RF output signal 406.

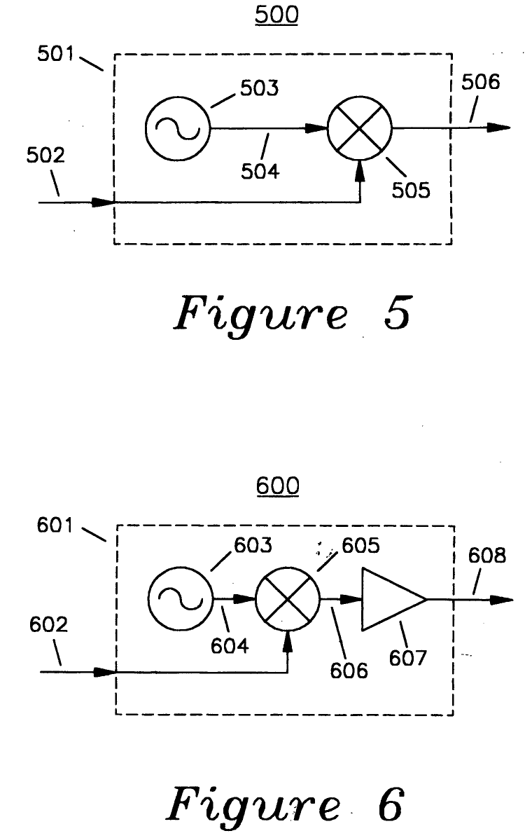

Other forms of modulation such as Amplitude Shift Key (ASK), On/Off Key (OOK) and Binary Phase Shift key (BPSK) require the addition of an amplitude modulator. The transmitter 501 shown schematically in FIG. 5 is capable of producing ASK, OOK, and BPSK modulated signals. A carrier generator 503, such as an oscillator, generates a carrier signal 504 which is coupled to an amplitude modulator 505 where it is modulated by an input modulating signal 502, which in the present invention is the digital data stream 118 from the processor 117 of FIG. 1, to produce an ASK, OOK, or BPSK modulated RF output signal 506. In some applications, the RF power produced by the transmitter of FIG. 5 is insufficient to provide reliable communications, and in such instances it may be suitable to include an amplifier stage to increase the output power. Such a transmitter is shown schematically as 601 in FIG. 6 where a carrier generator 603, such as an oscillator, generates a carrier signal 604 which is coupled to an amplitude modulator 605 where it is modulated by a modulating signal 602, which in the present invention is the digital data stream 118 from the processor 117 of FIG. 1, producing an ASK, OOK, or BPSK modulated RF signal 606 which is then amplified by a power amplifier 607, producing an amplified ASK, OOK, or BPSK modulated RF output signal 608.

Those familiar with the art will recognise that the transmitter power efficiency of the frequency modulated transmitter 301 of FIG. 3 and the amplitude modulated transmitter 501 of FIG. 5 can be improved by coupling the modulated RF output signal 121 of FIG. 1 to the antenna 122 of FIG. 1 by way of a Class E or Class F network. Those familiar with the art will also recognise that the transmitter power efficiency of the amplified frequency modulated transmitter 401 of FIG. 4 can be improved by using a Class C, Class E, or Class F for amplifier 405. Similarly, those familiar with the art will also recognise that the transmitter power efficiency of the amplified amplitude modulated transmitter 601 of FIG. 6 can be improved by using a Class C, Class E, or Class F for amplifier 607.

For applications using sufficiently low carrier frequencies, a microcontroller can perform the functions of carrier signal generation and modulation, making it entirely possible to realise the converter 111, processor 117, and transmitter 120 of FIG. 1 in a single microcontroller 123, yielding an extremely small and power efficient wireless biological monitoring instrument. Further, the power amplifier 405 of FIG. 4 and the power amplifier 607 of FIG. 6 may be realised by way of producing the RF output signal 121 of FIG. 1 differentially, which results in a 6 dB increase in RF output power. This is an efficient and cost effective method of realising an amplifier circuit that is commonly known as a bridge amplifier. Making use of such a method of amplification yields a cost effective biological monitoring instrument of increased range that is both physically small and power efficient.

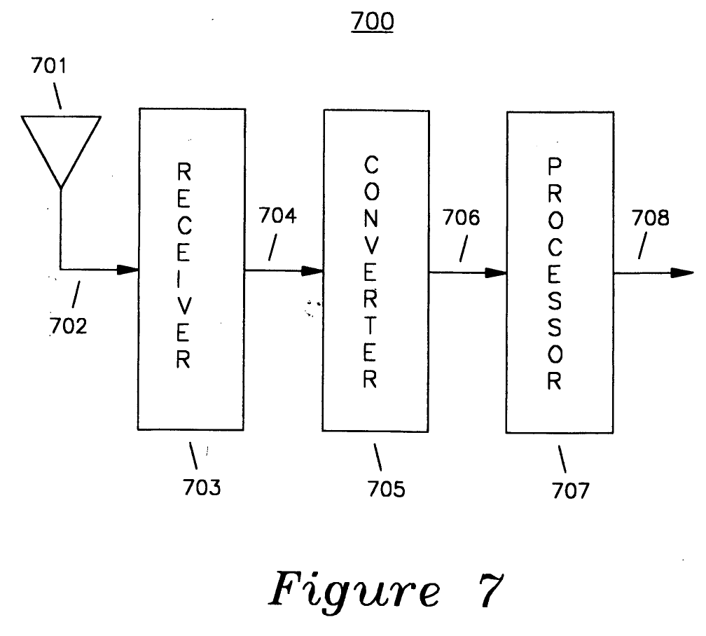

Some applications may require remote manipulation of the instrument, such as for calibration of the various sensors. For this purpose, a receiver for receiving signals from a remote transmitter may be included in the instrument, and such a receiver is described schematically in FIG. 7. Here, an antenna 701 receives signals 702 from a remote transmitter and couples the signals to a receiver 703, which produces a demodulated output signal 704. This demodulated signal is then coupled to a converter 705, such as a bit slicer, which produces a received output digital signal 706 which is coupled to a processor 707. Processor 707 decodes the received digital signal, sending instructions 708 to the instrument for the purpose of executing functions in response to the received digital signal. In practice, the function of processor 707 may be provided by the processor 117 of the microcontroller 123 of FIG. 1. Further, some microcontrollers may provide means for realising the converter 705 of FIG. 7, such as a voltage comparator, which will provide a cost effective method for including the receiver 700 in the instrument.

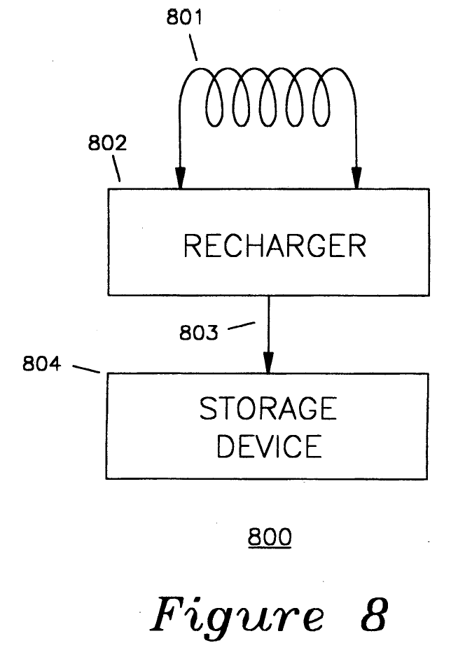

In certain applications, the consumption of electrical power by the instrument may be such that the power source 114 of FIG. 1 may be provided by a battery. In certain applications, one or more sensors may have a serviceable lifetime such that the sensor or sensors will expire before the battery. Still other applications may require that a rechargeable source of electrical power be provided, such applications including situations in which battery size is limited due to overall size limitations or where it is intended that the instrument be used repeatedly for short periods of time. Further, the mechanical nature of the instrument, which is to be discussed later, is such that the periodic replacement of expendables such as batteries is not possible. In such applications, the present invention may include a rechargeable source of electrical power for the power source 114 of FIG. 1, and such a rechargeable power source is illustrated schematically in FIG. 8. Here, a rechargeable electrical power storage device 804, such as a battery or a capacitor, receives a charging current 803 from a recharging circuit 802. The recharging circuit 802 is coupled to a remote source of electrical power by way of a coupling device 801, which is shown in FIG. 8 as being an inductive pickup loop, which is commonly used in practice for the realisation of contactless recharging devices.

The general nature of biological parameter monitoring instruments such as the present invention is that they are to be used inside the human or animal subject, which is commonly referred to as in vivo. It is well known by those familiar with the art that the operating environment of in vivo instruments such as the present invention is hostile to electronic devices and therefore a means of protecting such instruments from the in vivo environment is necessary. At the same time, the human or animal subject needs to be insulated from electrical voltages and possibly chemicals used in the various biological parameter sensors that may be detrimental to the health of the human or animal subject. Additionally, the human or animal subject needs to be protected from sharp edges and projections that may be injurious. To this effect, it is intended that the present invention be fully encapsulated in a moulding material, such as a resin or epoxy, that is both electrically insulating and inert to chemicals both inside and outside the instrument. It is also the intention of the present invention that the exterior surface of the encapsulation be entirely free of sharp edges and projections. Such a method of encapsulation suggests a variety of physical forms, one of which is that of a large pill, commonly referred to as a bolus, which is an elongated shape having rounded edges and ends.

Regardless as to whether the present invention is to be used for either short-term or long-term monitoring applications, the assemblage of the housing by way of multiple pieces and sealing gaskets is seen to be impractical, as such assemblies cannot fully guarantee that the seals will not ultimately fail and be breached, resulting in damage or failure of the instrument and risking injury to the subject. Therefore, it is intended that the housing of the present invention is to consist of a one-piece encapsulation of the aforementioned moulding material, and that such encapsulation will have features that will allow for the various biological sensors to make physical contact with the monitored environment whenever necessary.

Certain applications of the present invention will require that the instrument be made such that it will be heavy or rather have a high specific gravity. Such an application would include an instrument that is to be ingested by a ruminant animal, such as a dairy cow, where the instrument is to remain in the rumen stomach for an extended period of time. For such applications, the encapsulating moulding material may be mixed with an inert material such as glass beads or ceramic power which will increase the density or specific gravity of the encapsulating moulding material and thereby increase the mass of the instrument.

1. A wireless instrument for monitoring a plurality of biological parameters comprising:
A plurality of sensor means for measuring a plurality of biological parameters;
A data conversion means which is coupled to the biological parameter sensor means for converting the said measured biological sensor information to a digital format;
A processing means which is coupled to the data conversion means for collecting the said digitised sensor data and forming a digital data stream that comprises the collected digitised sensor data;
A transmitting means which modulates a radio frequency carrier signal with the said digital data stream to form a modulated radio frequency signal;
An antenna means which is coupled to the transmitting means to create a radiating electromagnetic field with the said modulated radio frequency signal;
An electrical power source that provides power for the said sensors, data conversion means, processing means, and transmitting means;
A microcontroller for providing the said data conversion means, the said processing means, and the said transmitting means; and
A sealed housing that is non-conductive, transparent to electromagnetic energy, and impervious to the monitored environment.

2. A wireless instrument for monitoring a plurality of biological parameters as claimed in claim 1 which further includes a means for monitoring operational parameters of the said instrument.

3. A wireless instrument for monitoring a plurality of biological parameters as claimed in claim 1 which further includes:
A receiving means for receiving signals from a remote transmitter; and
A conversion means for converting the said received signals into a digital data stream.

4. A wireless instrument for monitoring a plurality of biological parameters as claimed in claim 1 wherein the data conversion means consists of a single analogue-to-digital converter and an analogue multiplexer for the purpose of coupling the plurality of sensors individually to the analogue-to-digital converter to produce a single digital output signal.

5. A wireless instrument for monitoring a plurality of biological parameters as claimed in claim 1 wherein the said transmitting means consists of a frequency modulated carrier signal generator which is modulated by the said digital data stream.

6. A wireless instrument for monitoring a plurality of biological parameters as claimed in claim 1 wherein the transmitting means consists of a carrier signal generator which is coupled to a modulation means which is modulated by the said digital data stream.

7. A wireless instrument for monitoring a plurality of biological parameters as claimed in claim 1 wherein the instrument further includes a power amplifier for the purpose of increasing the transmitted signal power.

8. A wireless instrument for monitoring a plurality of biological parameters as claimed in claim 7 wherein the power amplifier consists of a differential RF output signal produced by the said microcontroller.

9. A wireless instrument for monitoring a plurality of biological parameters as claimed in claim 1 wherein the said electrical power source consists of a battery.

10. A wireless instrument for monitoring a plurality of biological parameters as claimed in claim 1 wherein the said electrical power source consists of a rechargeable electrical storage device that is charged by coupling to an external power means.

11. A wireless instrument for monitoring a plurality of biological parameters as claimed in claim 1 wherein the said digital data stream further includes an identification number.

12. A wireless instrument for monitoring a plurality of biological parameters as claimed in claim 1 wherein the said digital data stream further includes data relating to the operational parameters of the said instrument.

13. A wireless instrument for monitoring a plurality of biological parameters as claimed in claim 1 wherein the said digital data stream further includes a means of error correction.

14. A wireless instrument for monitoring a plurality of biological parameters as claimed in claim 1 wherein the said encapsulation includes a ceramic powder for the purpose of increasing the mass of the instrument.

15. A wireless instrument for monitoring a plurality of biological parameters as claimed in claim 1 wherein the said encapsulation includes a glass powder for the purpose of increasing the mass of the instrument.

16. A method for remotely monitoring a plurality of biological parameters which includes the steps of:
Measuring a plurality of biological parameters by way of a plurality of sensor means;
Converting the said plurality of measured biological parameters from the said plurality of sensor means to a digital format by way of a conversion means;
Combining the said plurality of digitised biological parameter sensor data into a digital data stream by way of a processing means;
Modulating a radio frequency carrier signal with the said digital data stream to produce a modulated radio frequency signal by way of a transmitting means; and
Coupling the said modulated radio frequency signal to a remote receiver by way of a radiating electromagnetic field produced by an antenna means.

17. The method of remotely monitoring a plurality of biological parameters as claimed in claim 16 in which the said conversion means for converting the said plurality of measured biological parameters from the said plurality of sensor means further includes a switch for the purpose of selecting the individual sensor means.

18. The method of remotely monitoring a plurality of biological parameters as claimed in claim 16 in which the said processing means, the said processing means, and the said transmitting means consists of a microcontroller.

19. The method of remotely monitoring a plurality of biological parameters as claimed in claim 16 in which the said transmitting means consists of a frequency modulated signal carrier generator.

20. The method of remotely monitoring a plurality of biological parameters as claimed in claim 16 which further includes a means for performing functions on demand by way of receiving signals from a remote transmitter which includes the steps of:
Receiving signals from a remote transmitter by way of a receiving means;
Demodulating the said received signals by way of a demodulation means;
Converting the said received demodulated signals into a digital data stream;
Conducting the said received digital data stream to the said processing means; and
Performing functions in accordance with instructions that are received in the said received digital data stream.
